(19) United States
(12) Patent Application Publication
(10) Pub. No.: US 2025/0177134 A1
(43) Pub. Date: Jun. 5, 2025

(54) AORTIC CROSSING CATHETER

(71) Applicant: BOSTON SCIENTIFIC SCIMED, INC., MAPLE GROVE, MN (US)

(72) Inventors: Gavin O'Brien, Dublin (IE); Tim O'Connor, Galway (IE); Chris Cullen, Galway (IE); John Lardner, Galway (IE)

(73) Assignee: BOSTON SCIENTIFIC SCIMED, INC., MAPLE GROVE, MN (US)

(21) Appl. No.: 18/969,864

(22) Filed: Dec. 5, 2024

Related U.S. Application Data
(60) Provisional application No. 63/606,363, filed on Dec. 5, 2023.

(51) Int. Cl. A61F 2/24

(52) U.S. Cl. CPC A61F 2/2436

(57) ABSTRACT

An aortic crossing catheter for directing a crossing wire through the aortic valve may include an outer sheath, an inner sheath slidably disposed within the outer sheath, and an expandable member at least partially fixed to the inner sheath and configured to move between a constrained delivery configuration to a radially expanded deployed configuration. Axial movement of the inner and outer sheaths relative one another radially expands the expandable member such that when a crossing wire is inserted through a lumen of the inner sheath, the wire is directed through the aortic valve.

### CROSS-REFERENCE TO RELATED APPLICATIONS

This application claims the benefit of priority of U.S. Provisional Application No. 63/606,363 filed Dec. 5, 2023, the entire disclosure of which is hereby incorporated by reference.

### TECHNICAL FIELD

The disclosure pertains to medical devices and more particularly to delivery systems for replacement heart valves, and methods for using such medical devices and systems.

### BACKGROUND

A wide variety of medical devices have been developed for medical use including, for example, medical devices utilized to replace heart valves. Heart function can be significantly impaired when a heart valve is not functioning properly. When the heart valve is unable to close properly, the blood within a heart chamber can regurgitate, or leak backwards through the valve. Valve regurgitation may be treated by replacing or repairing a diseased valve, such as an aortic valve. Surgical valve replacement is one method for treating the diseased valve, however this requires invasive surgical openings into the chest cavity and arresting of the patient's heart and cardiopulmonary bypass. Minimally invasive methods of treatment, such as transcatheter aortic valve implantation (TAVI) or transcatheter aortic valve replacement (TAVR), generally involve the use of delivery catheters that are delivered through arterial passageways or other anatomical routes into the heart to replace the diseased valve with an implantable prosthetic heart valve.

In most cases, accessing a heart valve for replacement requires placement of a guidewire through the valve leaflets. Of the known delivery devices and methods for accessing a heart valve for replacement, each has certain advantages and disadvantages. There is an ongoing need to provide alternative devices and methods for manufacturing and using the medical devices.

### SUMMARY

This disclosure provides design, material, manufacturing method, and use alternatives for medical devices. An example aortic crossing catheter includes an outer sheath, an inner sheath slidably disposed within the outer sheath, and an expandable member at least partially fixed to the inner sheath and configured to move between a constrained delivery configuration to a radially expanded deployed configuration, wherein axial movement of the inner and outer sheaths relative one another radially expands the expandable member.

Alternatively or additionally to the embodiment above, a proximal end of the expandable member is fixed to a distal end of the outer sheath and a distal end of the expandable member is fixed to the inner sheath, wherein proximal movement of the inner sheath relative to the outer sheath radially expands the expandable member.

Alternatively or additionally to any of the embodiments above, the distal end of the expandable member is fixed to a distal end of the inner sheath.

Alternatively or additionally to any of the embodiments above, the expandable member has a heat set expanded shape with a cylindrical body and conical ends.

Alternatively or additionally to any of the embodiments above, the expandable member has a heat set expanded shape with a disk-shaped body and cylindrical ends.

Alternatively or additionally to any of the embodiments above, the expandable member has an adjustable outer diameter.

Alternatively or additionally to any of the embodiments above, the expandable member is configured to have an outer diameter of between 10 mm and 35 mm in an expanded configuration.

Alternatively or additionally to any of the embodiments above, the expandable member is configured to have a length of between 5 mm and 30 mm in the expanded configuration.

Alternatively or additionally to any of the embodiments above, the distal end of one or both of the outer sheath and the inner sheath includes a radiopaque marker.

Alternatively or additionally to any of the embodiments above, the radiopaque marker is a parallax marker.

Alternatively or additionally to any of the embodiments above, the expandable member is self-expandable, wherein a proximal end of the expandable member is fixed to the inner sheath and a distal end of the expandable member is coupled to the inner sheath, wherein movement of the outer sheath proximally off the expandable member allows the expandable member to radially expand.

Alternatively or additionally to any of the embodiments above, the distal end of the expandable member is fixed to the inner sheath.

Alternatively or additionally to any of the embodiments above, the distal end of the expandable member is fixed to a collar freely slidable over the inner sheath.

Alternatively or additionally to any of the embodiments above, the expandable member has a heat set expanded shape with a cylindrical body and conical ends.

Alternatively or additionally to any of the embodiments above, the expandable member has a heat set expanded shape with a disk-shaped body and cylindrical ends.

Alternatively or additionally to any of the embodiments above, the expandable member is configured to have an outer diameter of between 10 mm and 35 mm in an expanded configuration.

Alternatively or additionally to any of the embodiments above, the expandable member is configured to have a length of between 5 mm and 30 mm in the expanded configuration.

Alternatively or additionally to any of the embodiments above, the distal end of one or both of the outer sheath and the inner sheath includes a radiopaque parallax marker.

Alternatively or additionally to any of the embodiments above, the aortic crossing catheter further includes a crossing wire disposed within the inner sheath. An example method of inserting a crossing wire through an aortic valve of a patient includes inserting an aortic crossing catheter across the patient's aortic arch and into the ascending aorta, the aortic crossing catheter having an expandable member attached thereto in a compressed configuration, expanding the expandable member, and inserting the crossing wire through the aortic crossing catheter and the expandable member and through the aortic valve.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1A illustrates one embodiment of an aortic crossing catheter in the aorta with an expanded member positioned in the sinotubular junction;

FIG. 1B is a top-down view of an example aortic valve;

FIG. 2 is a side cross-sectional view of the aortic crossing catheter of FIG. 1A;

FIG. 3A is a side view of an example aortic crossing catheter;

FIG. 3B is a side view of another example aortic crossing catheter;

FIGS. 4A and 4B illustrate adjusting the angle of the crossing wire;

FIGS. 5A and 5B illustrate a parallax marker;

FIG. 6 is a side cross-sectional view of another example aortic crossing catheter; and

FIGS. 7A-7C illustrate steps in expanding an example aortic crossing catheter.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

Performing transcatheter aortic valve replacement (TAVR) procedures requires crossing the native or previously implanted prosthetic valve with a crossing wire. Access can be difficult due to interaction of the crossing wire with the diseased native aortic valve leaflets. In cases where calcification, aortic stenosis and bicuspid valves are present, the difficulty in crossing the valve increases. This difficulty may result in increased procedural time, the use of multiple accessories, increased exposure to fluoroscopy and radiation for the patient, and frustration for the physician. In some cases the procedure may be postponed due to the amount of time taken to complete this initial task.

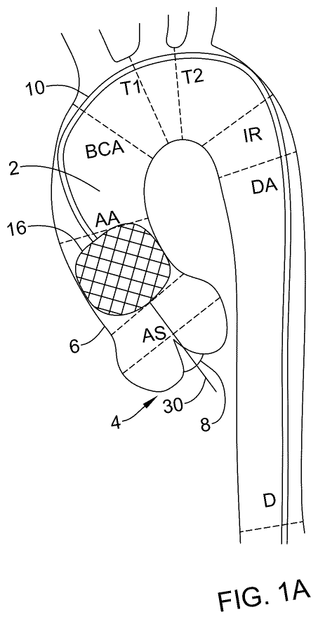

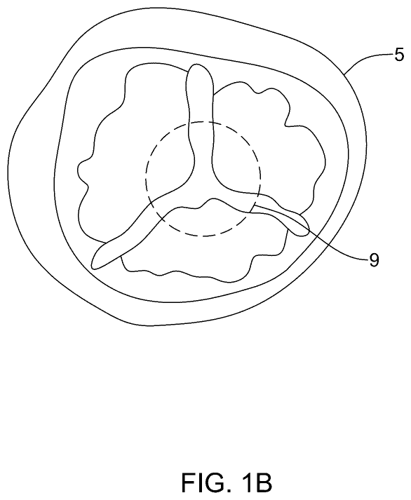

An aortic crossing catheter 10 may be delivered through the aorta from the level of the diaphragm (D), through the descending aorta (DA), isthmic region (IR), across the region between the origin of the left common carotid artery and the left subclavian artery (T2), the region between the origin of the brachiocephalic artery and the left common carotid artery (T1), the region proximal to the origin of the brachiocephalic artery BCA, and into the ascending aorta (AA) 2, as shown in FIG. 1A. The crossing catheter 10 positions the crossing wire 30 in a central position within the ascending aorta 2 in relation to the annulus 4, above the aortic sinus (AS) and concentric to the sinotubular junction (STJ) 6, as shown in FIG. 1A. This centering function may be achieved with a permeable expandable member 16 disposed on the aortic crossing catheter 10, with the expanded member filling the aorta at the STJ and positioning a lumen extending through the member in alignment with the native or prosthetic aortic valve 8. With the aortic crossing catheter 10 positioned centrally in the STJ 6, the catheter lumen is concentric with the native aorta and aortic valve, and a crossing wire 30 can be fed through the catheter lumen, crossing through the native or prosthetic valve. As shown in FIG. 1B, the desired location for inserting the crossing wire 30 is through the middle region 9 of the aortic valve 5, where all three of the valve leaflets come together. Once the crossing wire is in position across the valve, the centering member may be recaptured within the sheath and removed, leaving the crossing wire in place. The TAVR procedure may then continue in a timely manner. The expandable member is permeable so there is no need for rapid pacing.

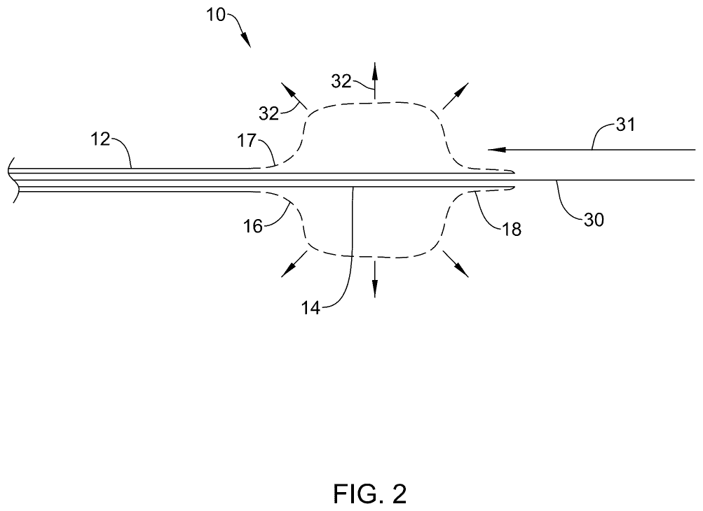

An example aortic crossing catheter 10 is illustrated in FIG. 2. In this embodiment, the catheter includes an outer sheath 12, an inner sheath 14 slidably disposed within a lumen in the outer sheath, and an expandable member 16 coupled to the inner and outer sheaths. The expandable member 16 is at least partially fixed to the inner sheath 14 and is configured to move between a constrained delivery configuration to a radially expanded deployed configuration. In some examples, the proximal end 17 of the expandable member 16 may be fixed to the distal end of the outer sheath 12 and the distal end 18 of the expandable member 16 may be fixed to the inner sheath 14. The aortic crossing catheter 10 may be delivered through a delivery catheter (not shown) in the constrained delivery configuration in which the expandable member 16 is elongated and compressed around the inner sheath 14. Once the aortic crossing catheter 10 is positioned within the ascending aorta, the delivery catheter may be withdrawn proximally. Thereafter, pulling or otherwise moving the inner sheath 14 proximally relative to the outer sheath 12, as indicated by arrow 31, radially expands the expandable member 16, as indicated by arrows 32. Alternatively, the outer sheath 12 may be pushed distally relative to the inner sheath 14. This axial movement of the inner and outer sheaths relative one another causes the expandable member 16 to expand and contract. The expandable member 16 may have an adjustable outer diameter, based on the degree to which the inner sheath 14 is moved proximally. This allows the user to control the amount of expansion and also the final outer diameter of the expandable member, which allows for the expandable member 16 to be expanded to fully engage the aorta, regardless of the size of the aorta. A crossing wire 30 may be inserted through a lumen in the inner sheath 14. Once the crossing wire has been advanced through the aortic valve, the expandable member 16 may be removed by first advancing the inner sheath 14 distally relative to the outer sheath 12 to elongate and radially compress the expandable member 16. The inner and outer sheaths may then be withdrawn proximally leaving the crossing wire 30 in place within the valve. In some embodiments, the distal end 18 of the expandable member 16 may be fixed to a distal end of the inner sheath 14, as shown in FIG. 2.

In another embodiment, both proximal and distal ends 17, 18 of the expandable member 16 may be fixed to the outer sheath 12, with the inner sheath omitted. In this embodiment, the expandable member 16 is heat set to self-expand when the delivery catheter is withdrawn proximally off the outer sheath 12 and expandable member 16. Once the crossing wire 30 has been inserted through the lumen of the outer sheath 12, the outer sheath and expandable member may be removed by pulling the outer sheath proximally into the delivery sheath, at which point, the expandable member will be compressed.

The expandable member 16 may be formed from a at least one filament or wire 15 that may be woven, braided, wound, knitted, and combinations thereof, to form the expandable member 16. In some embodiments, the expandable member may be a woven or braided mesh. Any of the woven, braided, wound, and/or knitted wires may include a variety of different cross-sectional shapes (e.g., oval, round, flat, square, etc.) and may be formed from metals and/or polymers, including shape memory metals and polymers. The woven, braided, wound, and/or knitted wire may further include a single wire woven, braided, or wound upon itself, or multiple wires woven, braided, or wound together. The expandable member 16 may include multiple wires 15 of a metal material, such as nitinol or nitinol-containing material, or another nickel-titanium alloy, for example. In some instances, the wires 15 may have a diameter of about 0.011 inches, for example.

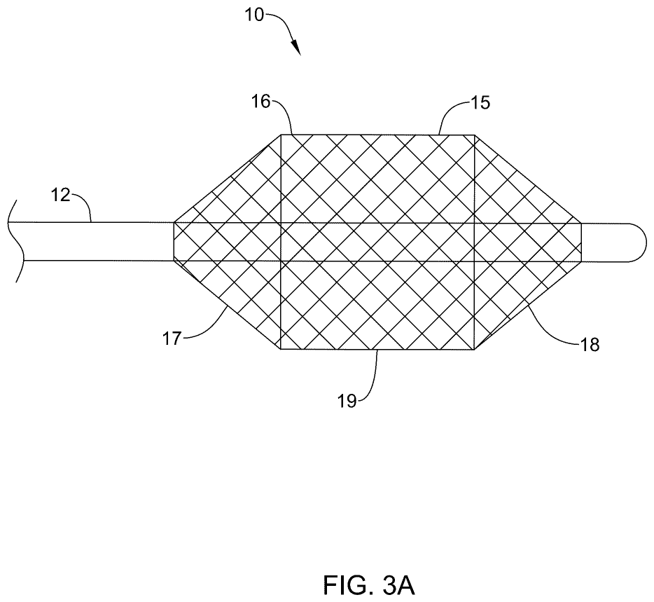

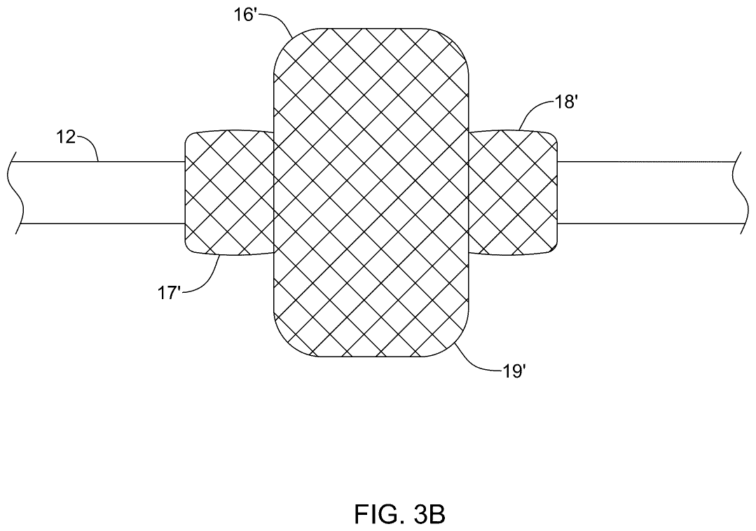

The expandable member 16 may be made of a shape memory material and may have a heat set expanded shape such that when the inner sheath 14 is pulled back proximally into the outer sheath 12, the expandable member expands gradually into the heat set shape. The general shape of the expandable member may be the same when the expandable member is slightly expanded up until it is fully expanded, with just the outer diameter changing. In some embodiments, the expandable member 16 may have a heat set expanded shape in which a body region 19 has a cylindrical shape and the proximal and distal ends 17, 18 each have a conical shape, as shown in FIG. 3A. The body region 19 may remain cylindrical as the outer diameter changes during expansion and contraction. The outer diameter of the body region 19 may be controlled by the position of the inner sheath 14 relative to the outer sheath 12. This structure of having a uniform shape during expansion may allow the user to expand the expandable member to fully engage the walls of the aorta to center the crossing wire regardless of the size of the aorta. The expandable member 16 may be gradually expanded to fit a wide range of aorta sizes, allowing the device to be used on patients of all ages.

In other embodiments, the expandable member 16′ may have a heat set expanded shape with a disk-shaped body region 19′ and cylindrical proximal and distal ends 17′, 18′, as shown in FIG. 3B. The body region 19′ may remain disk shaped as its outer diameter changes during expansion and contraction. The outer diameter of the body region 19 may be controlled by the position of the inner sheath 14 relative to the outer sheath 12. The body region 19′ may be gradually expanded to fit a wide range of aorta sizes.

The expandable members 16, 16′ may be expandable to have an outer diameter of the body region 19, 19′ of between 10 mm and 35 mm in a fully expanded configuration. In the fully compressed or contracted configuration, the outer diameter may be slightly larger than the outer diameter of the inner sheath 14. The length of the expandable member 16, particularly the body region 19 that is in contact with the inner wall of the aorta, must be sufficient to ensure concentricity within the aorta. In some embodiments, the body region 19 may have a length of between 5 mm and 30 mm in the expanded configuration.

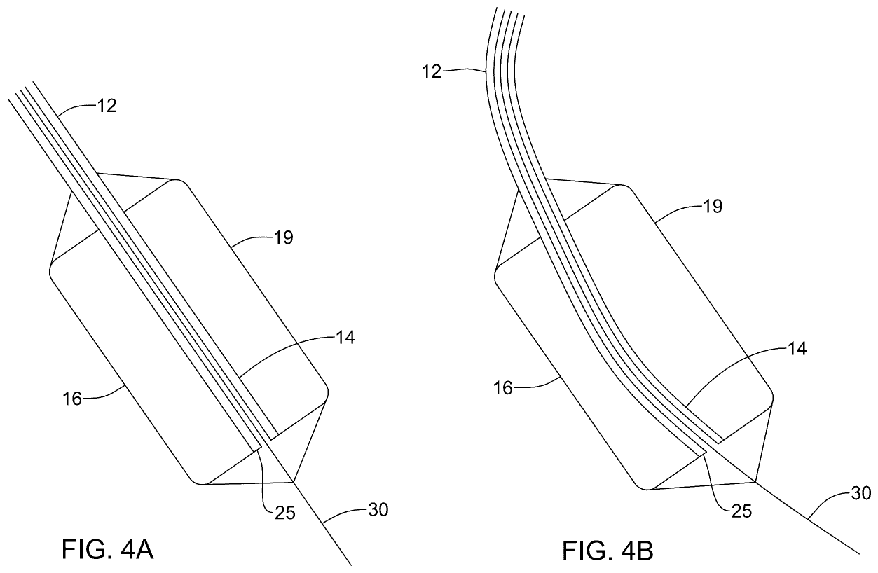

With the expandable member 16 in the fully expanded configuration and the body region 19 pressing against the inner wall of the aorta in the SJT, the crossing wire may be delivered through the lumen of the inner sheath 14. FIG. 4A illustrates the expanded body region 19 aligned with the inner and outer sheaths 14, 12 and the crossing wire 30, such that all elements have a single longitudinal axis. However, in some embodiments, the anatomy of the heart may be such that the angle of the crossing wire is desired to be adjusted. For example, a slight angle relative to the longitudinal axis of the expanded body region may be desired for passing the crossing wire 30 through the valve. This may be achieved by applying a push or pull force on the outer sheath 12 when the body region 19 is anchored in the aorta, resulting in the distal end of the inner sheath 14 bending at a slight angle, thereby directing the crossing wire 30 at an angle relative to the longitudinal axis of the expanded body region, as shown in FIG. 4B.

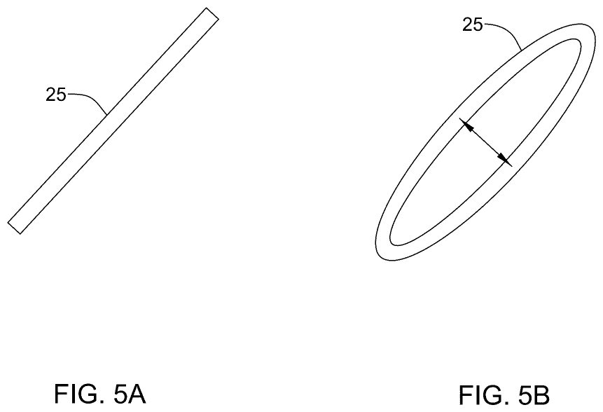

The angle of the crossing wire 30 relative to the aorta and valve may determine whether the operator is successful in getting the crossing wire through the valve. As the crossing wire 30 will be aligned with the distal end of the inner sheath 14, it will be desired to position the distal end of the inner sheath 14 in alignment with the aorta and valve. In order to determine whether the distal end of the inner sheath 14 is in the desired location and also at the desired angle relative to the valve, the distal end of one or both of the outer sheath 12 and inner sheath 14 may include a radiopaque marker. When the radiopaque marker is in the shape of a ring, the marker functions as a radiopaque parallax marker 25. FIGS. 5A and 5B illustrate the parallax marker 25. As shown in FIG. 5A, when the longitudinal axis of inner sheath is aligned with the longitudinal axis of the aorta and valve, the ring-shaped marker 25 on the distal end of the inner sheath is visible as a line. However, when the distal end of the inner sheath is out of alignment relative to the aorta and valve, the ring-shaped marker 25 is visible as an oval, as shown in FIG. 5B.

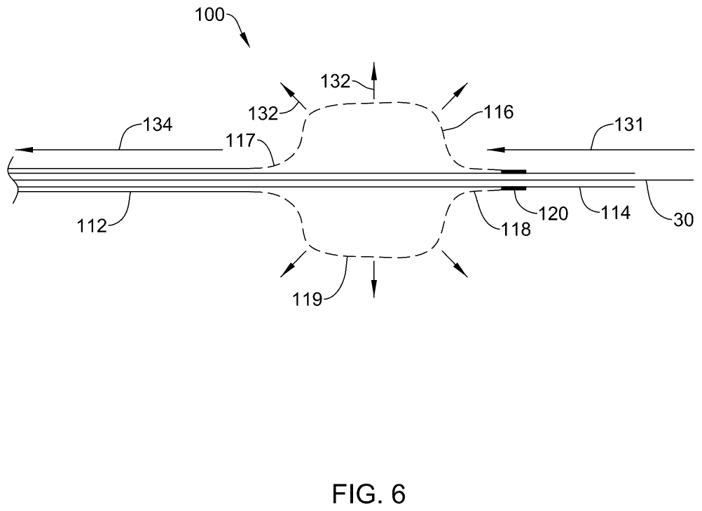

Another embodiment of aortic crossing catheter 100 is illustrated in FIG. 6. In this embodiment, the catheter includes an outer sheath 112, an inner sheath 114 slidably disposed within a lumen in the outer sheath, and an expandable member 16 coupled to the inner sheath 114. The proximal end 117 of the expandable member 116 may be fixed to the inner sheath 114 and the distal end 118 of the expandable member 116 may be coupled to the inner sheath 114. The expandable member 116 may have a heat set expanded shape with a cylindrical body region 119 and conical ends 117, 118. In other embodiments, the expandable member may have a heat set expanded shape with a disk-shaped body and cylindrical ends.

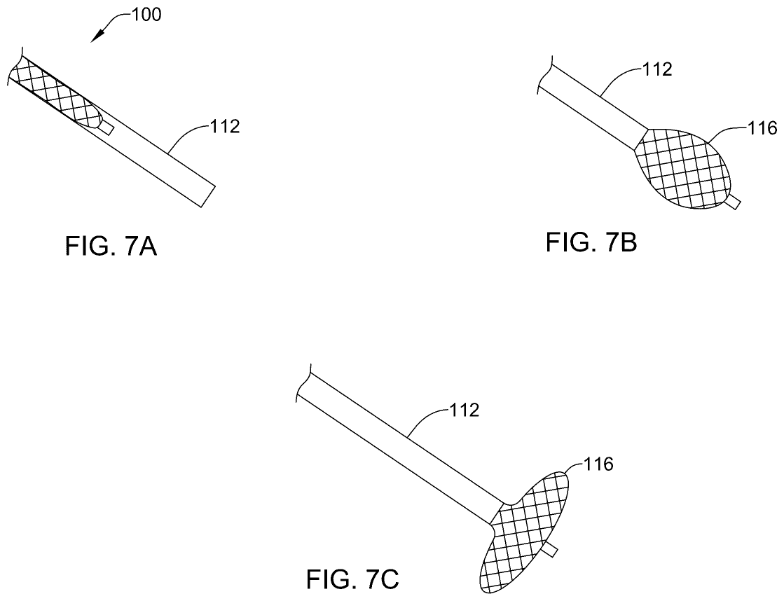

In the embodiment shown in FIG. 6, the proximal end 117 of the expandable member 116 may be fixed to the inner sheath 114 and the distal end 118 of the expandable member 116 may be fixed to a collar 120 freely moveable over the inner sheath 114. The aortic crossing catheter 100 may be delivered through a delivery catheter (not shown) with the outer sheath 112 disposed over the expandable member 116, holding it in an elongated, compressed configuration, as shown in FIG. 7A.

The expandable member 116 may be expandable to have an outer diameter of the body region 119 of between 10 mm and 35 mm in a fully expanded configuration. In the fully compressed or contracted configuration, the outer diameter may be slightly larger than the outer diameter of the inner sheath 114. The length of the expandable member 116, particularly the body region 119 that is in contact with the inner wall of the aorta, must be sufficient to ensure concentricity within the aorta. In some embodiments, the body region 119 may have a length of between 5 mm and 30 mm in the expanded configuration. The inner sheath 114 may have a ring-shaped radiopaque marker, such as the parallax marker discussed above.

Once the aortic crossing catheter 100 is positioned within the ascending aorta, the delivery catheter may be withdrawn proximally, leaving the aortic crossing catheter 100 in place in the compressed or contracted configuration as shown in FIG. 7A. Thereafter, pulling the outer sheath 112 proximally off of the expandable member 116, as indicated by arrow 134 in FIG. 6, allows the self-expanding expandable member 116 to begin radially expanding, as indicated by arrows 132 and shown in FIG. 7B. Radial expansion of the expandable member causes the collar 120 to move proximally over the inner sheath 114, as indicated by arrow 131. When the outer sheath 112 has been removed completely, the expandable member 116 expands to its fully expanded configuration, as shown in FIG. 7C. A crossing wire 30 may be inserted through a lumen in the inner sheath 114. Once the crossing wire has been advanced through the aortic valve, the expandable member 116 may be removed by first advancing the outer sheath 112 distally over the expandable member 116 to elongate and radially compress the expandable member 116, moving the collar 120 distally. The aortic crossing catheter 100 may then be withdrawn proximally leaving the crossing wire 30 in place within the valve.

In another embodiment, both proximal and distal ends 117, 118 of the expandable member 116 may be fixed to the inner sheath 114. In this embodiment, the expandable member 116 is heat set to self-expand when the outer sheath 112 is withdrawn proximally off the inner sheath 114 and expandable member 116. Once the crossing wire 30 has been inserted through the lumen of the inner sheath 114, the aortic crossing catheter 100 may be removed by first pushing the outer sheath 112 distally to compress the expandable member 116, and then removing the entire aortic crossing catheter 100 through the delivery catheter, leaving the crossing wire 30 in place within the valve.

The expandable member 116 may be formed from at least one filament or wire that may be woven, braided, wound, knitted, and combinations thereof, to form the expandable member 116, as described above with regard to the expandable member 16.

Any of the embodiments of crossing wire catheter described above may be used in a method of inserting a crossing wire through an aortic valve, such as in the preliminary steps for a

TAVR procedure. The method may include the steps of inserting the aortic crossing catheter 10, 100 across the aortic arch and into the ascending aorta, expanding the expandable member, and inserting a crossing wire through the aortic crossing catheter and the expandable member and through the aortic valve.

Once the crossing wire 30 has been delivered through the aortic valve using one of the aortic crossing catheters 10, 100 described above, a stent-valve delivery system may be advanced over the crossing wire. Following the advancement of the crossing wire 30 through the aortic valve, various elements of a delivery system are advanced along the crossing wire to replace a heart valve. In some embodiments, the delivery system is replacing a native heart valve. In other embodiments the delivery system is replacing a previously implanted heart valve. The delivery system may be inserted through a catheter through the femoral artery and vasculature, across the aortic arch and through the aortic valve.

It will be understood that the dimensions described in association with the above figure are illustrative only, and that other dimensions are contemplated. The materials that can be used for the various components of the system for use in replacement heart-valve implant procedures (and/or other systems or components disclosed herein) and the various elements thereof disclosed herein may include those commonly associated with medical devices. For simplicity purposes, the following discussion refers to the system for use in replacement heart-valve implant procedures (and variations, systems or components disclosed herein). However, this is not intended to limit the devices and methods described herein, as the discussion may be applied to other elements, members, components, or devices disclosed herein.

In some embodiments, the system for use in replacement heart-valve implant procedures (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super clastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-clastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear clastic and/or non-super-clastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-clastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the system for use in replacement heart-valve implant procedures (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the system for use in replacement heart-valve implant procedures (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system for use in replacement heart-valve implant procedures (and variations, systems or components thereof disclosed herein) to achieve the same result.

Useful and nonlimiting examples of polymeric materials for making the expandable member 16 include poly (L-lactide) (PLLA), poly (D,L-lactide) (PLA), poly (glycolide) (PGA), poly (L-lactide-co-D,L-lactide) (PLLA/PLA), poly (L-lactide-co-glycolide) (PLLA/PGA), poly (D,L-lactide-coglycolide) (PLA/PGA), poly (glycolide-co-trimethylene carbonate)

(PGA/PTMC), polydioxanone (PDS), Polycaprolactone (PCL), polyhydroxybutyrate (PHBT), poly (phosphazene) poly (D,L-lactide-co-caprolactone) PLA/PCL), poly (glycolide-co-caprolactone) (PGA/PCL), poly (phosphate ester) and the like. Wires 15 made from polymeric materials may also include radiopaque materials, such as metallic-based powders, particulates or pastes which may be incorporated into the polymeric material. For example the radiopaque material may be blended with the polymer composition from which the polymeric wire is formed, and subsequently fashioned into the expandable member 16 as described herein. Alternatively, the radiopaque material may be applied to the surface of the metal or polymer wire 15 of the expandable member 16. In either embodiment, various radiopaque materials and their salts and derivatives may be used including, without limitation, bismuth, barium and its salts such as barium sulphate, tantalum, tungsten, gold, platinum and titanium, to name a few. Additional useful radiopaque materials may be found in U.S. Pat. No. 6,626,936, the contents of which are incorporated herein by reference. Metallic complexes useful as radiopaque materials are also contemplated. The expandable member 16 may be selectively made radiopaque at desired areas along the wire or may be fully radiopaque. In some instances, the wires 15 may have a composite construction having an inner core of tantalum, gold, platinum, tungsten, iridium or combination thereof and an outer member or layer of nitinol to provide a composite wire for improved radiopacity or visibility. In one example, the inner core may be platinum and the outer layer may be nitinol. The inner core of platinum may represent about at least 10% of the wire 15 based on the overall cross-sectional percentage. Moreover, nitinol that has not been treated for shape memory such as by heating, shaping and cooling the nitinol at its martensitic and austenitic phases, is also useful as the outer layer. Further details of such composite wires may be found in U.S. Pat. No. 7,101,392, the contents of which is incorporated herein by reference. The wires 15 may be made from nitinol, or a composite wire having a central core of platinum and an outer layer of nitinol. Further, the filling weld material, if required by welding processes such as MIG, may also be made from nitinol, stainless steel, cobalt-based alloy such as ELGILOY®, platinum, gold, titanium, tantalum, niobium, and combinations thereof.

In some embodiments, the system for use in replacement heart-valve implant procedures (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated cthylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An aortic crossing catheter, comprising:
an outer sheath;
an inner sheath slidably disposed within the outer sheath; and
an expandable member at least partially fixed to the inner sheath and configured to move between a constrained delivery configuration to a radially expanded deployed configuration, wherein axial movement of the inner and outer sheaths relative one another radially expands the expandable member.

2. The aortic crossing catheter of claim 1, wherein a proximal end of the expandable member is fixed to a distal end of the outer sheath and a distal end of the expandable member is fixed to the inner sheath, wherein proximal movement of the inner sheath relative to the outer sheath radially expands the expandable member.

3. The aortic crossing catheter of claim 2, wherein the distal end of the expandable member is fixed to a distal end of the inner sheath.

4. The aortic crossing catheter of claim 2, wherein the expandable member has a heat set expanded shape with a cylindrical body and conical ends.

5. The aortic crossing catheter of claim 2, wherein the expandable member has a heat set expanded shape with a disk-shaped body and cylindrical ends.

6. The aortic crossing catheter of claim 2, wherein the expandable member has an adjustable outer diameter.

7. The aortic crossing catheter of claim 6, wherein the expandable member is configured to have an outer diameter of between 10 mm and 35 mm in an expanded configuration.

8. The aortic crossing catheter of claim 7, wherein the expandable member is configured to have a length of between 5 mm and 30 mm in the expanded configuration.

9. The aortic crossing catheter of claim 2, wherein the distal end of one or both of the outer sheath and the inner sheath includes a radiopaque marker.

10. The aortic crossing catheter of claim 9, wherein the radiopaque marker is a parallax marker.

11. The aortic crossing catheter of claim 1, wherein the expandable member is self-expandable, wherein a proximal end of the expandable member is fixed to the inner sheath and a distal end of the expandable member is coupled to the inner sheath, wherein movement of the outer sheath proximally off the expandable member allows the expandable member to radially expand.

12. The aortic crossing catheter of claim 11, wherein the distal end of the expandable member is fixed to the inner sheath.

13. The aortic crossing catheter of claim 11, wherein the distal end of the expandable member is fixed to a collar freely slidable over the inner sheath.

14. The aortic crossing catheter of claim 11, wherein the expandable member has a heat set expanded shape with a cylindrical body and conical ends.

15. The aortic crossing catheter of claim 11, wherein the expandable member has a heat set expanded shape with a disk-shaped body and cylindrical ends.

16. The aortic crossing catheter of claim 11, wherein the expandable member is configured to have an outer diameter of between 10 mm and 35 mm in an expanded configuration.

17. The aortic crossing catheter of claim 16, wherein the expandable member is configured to have a length of between 5 mm and 30 mm in the expanded configuration.

18. The aortic crossing catheter of claim 11, wherein the distal end of one or both of the outer sheath and the inner sheath includes a radiopaque parallax marker.

19. The aortic crossing catheter of claim 11, further comprising a crossing wire disposed within the inner sheath.

20. A method of inserting a crossing wire through an aortic valve of a patient, comprising:
inserting an aortic crossing catheter across the patient's aortic arch and into the ascending aorta, the aortic crossing catheter having an expandable member attached thereto in a compressed configuration;
expanding the expandable member; and
inserting the crossing wire through the aortic crossing catheter and the expandable member and through the aortic valve.
